(19) United States
(12) Patent Application Publication
(10) Pub. No.: US 2022/0028503 A1
(43) Pub. Date: Jan. 27, 2022

(54) DYNAMIC PATIENT OUTCOME PREDICTIONS

(71) Applicant: International Business Machines Corporation, Armonk, NY (US)

(72) Inventors: Brendan Bull, Durham, NC (US); Paul Lewis Felt, Springville, UT (US); Mario J. Lorenzo, Miami, FL (US)

(21) Appl. No.: 16/936,500

(22) Filed: Jul. 23, 2020

(51) Int. Cl. G16H 10/60; G16H 50/70; G16H 40/63; G16H 40/67

(52) U.S. Cl. CPC G16H 10/60; G16H 40/67; G16H 40/63; G16H 50/70

(57) ABSTRACT

Aspects of the present disclosure include receiving, by a processor, patient medical data associated with a patient, analyzing the patient medical data to generate a present patient summary for the patient, determining a cohort for the patient based on the present patient summary, the cohort comprising a plurality of similarly situated patients, and generating at least one medical prediction for the patient at a future age group based at least in part on the cohort for the patient.

### BACKGROUND

The present invention generally relates to patient summarization, and more specifically, to dynamic patient outcome predictions.

An Electronic Medical Record (EMR), or Electronic Health Record, is a digital record of a patient's medical history. An EMR tracks a patient's medical history over time and may include a range of data including both unstructured and structure data. Examples of unstructured data include notes by a variety of medical care providers, for example clinician notes. Examples of structured data include procedures performed, lab results, and medications taken. Over time the amount of information in a patient's EMR can becomes very large and summarization of this data can be difficult. Accordingly, there is a need for summarization of this large data set for various patients to assist with prediction patient outcomes.

### SUMMARY

Embodiments of the present invention are directed to a computer-implemented method for dynamic patient outcome predictions. A non-limiting example of the computer-implemented method includes receiving, by a processor, patient medical data associated with a patient, analyzing the patient medical data to generate a present patient summary for the patient, determining a cohort for the patient based on the present patient summary, the cohort comprising a plurality of similarly situated patients, and generating at least one medical prediction for the patient at a future age group based at least in part on the cohort for the patient.

Embodiments of the present invention are directed to a system for dynamic patient outcome predictions. A non-limiting example of the system includes a processor coupled to a memory, the processor configured to perform receiving, by a processor, patient medical data associated with a patient, analyzing the patient medical data to generate a present patient summary for the patient, determining a cohort for the patient based on the present patient summary, the cohort comprising a plurality of similarly situated patients, and generating at least one medical prediction for the patient at a future age group based at least in part on the cohort for the patient.

Embodiments of the invention are directed to a computer program product for dynamic patient outcome predictions, the computer program product comprising a computer readable storage medium having program instructions embodied therewith. The program instructions are executable by a processor to cause the processor to perform a method. A non-limiting example of the method includes receiving, by a processor, patient medical data associated with a patient, analyzing the patient medical data to generate a present patient summary for the patient, determining a cohort for the patient based on the present patient summary, the cohort comprising a plurality of similarly situated patients, and generating at least one medical prediction for the patient at a future age group based at least in part on the cohort for the patient.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a cloud computing environment according to one or more embodiments of the present invention;

FIG. 2 depicts abstraction model layers according to one or more embodiments of the present invention;

FIG. 3 depicts a block diagram of a computer system for use in implementing one or more embodiments of the present invention;

FIG. 4 depicts block diagram of a system for dynamic patient outcomes predictions according to embodiments of the invention; and

FIG. 5 depicts a flow diagram of a method for dynamic patient outcome predictions according to one or more embodiments of the invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

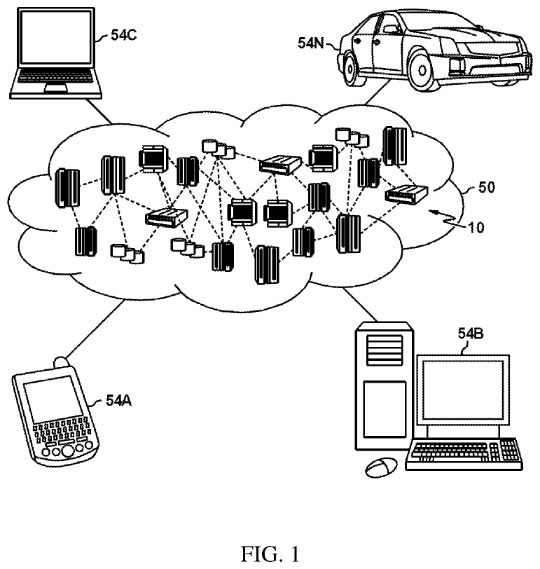

Referring now to FIG. 1, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that computing nodes 10 and cloud computing environment 50 can communicate with any type of computerized device over any type of network and/or network addressable connection (e.g., using a web browser).

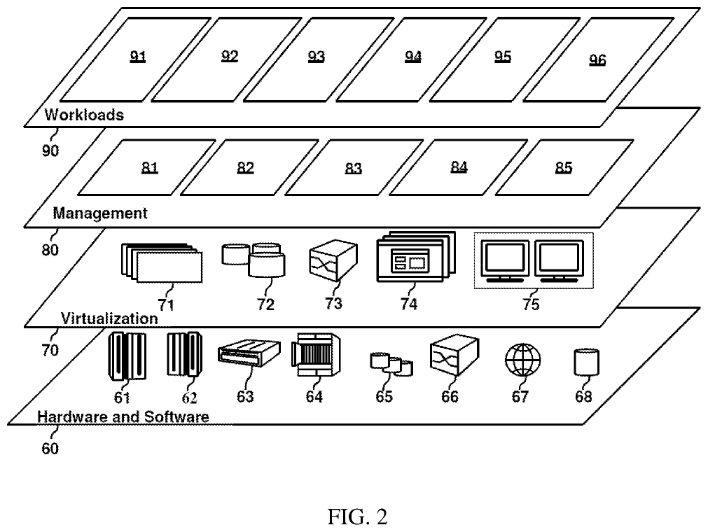

Referring now to FIG. 2, a set of functional abstraction layers provided by cloud computing environment 50 (FIG. 1) is shown. It should be understood in advance that the components, layers, and functions shown in FIG. 2 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provides pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and dynamic patient outcome predictions 96.

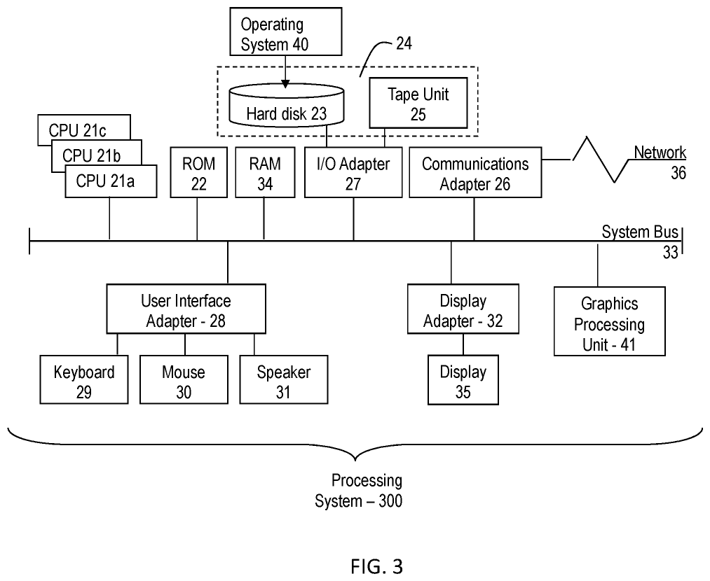

Referring to FIG. 3, there is shown an embodiment of a processing system 300 for implementing the teachings herein. In this embodiment, the system 300 has one or more central processing units (processors) 21a, 21b, 21c, etc. (collectively or generically referred to as processor(s) 21). In one or more embodiments, each processor 21 may include a reduced instruction set computer (RISC) microprocessor. Processors 21 are coupled to system memory 34 and various other components via a system bus 33. Read only memory (ROM) 22 is coupled to the system bus 33 and may include a basic input/output system (BIOS), which controls certain basic functions of system 300.

FIG. 3 further depicts an input/output (I/O) adapter 27 and a network adapter 26 coupled to the system bus 33. I/O adapter 27 may be a small computer system interface (SCSI) adapter that communicates with a hard disk 23 and/or tape storage drive 25 or any other similar component. I/O adapter 27, hard disk 23, and tape storage device 25 are collectively referred to herein as mass storage 24. Operating system 40 for execution on the processing system 300 may be stored in mass storage 24. A network adapter 26 interconnects bus 33 with an outside network 36 enabling data processing system 300 to communicate with other such systems. A screen (e.g., a display monitor) 35 is connected to system bus 33 by display adaptor 32, which may include a graphics adapter to improve the performance of graphics intensive applications and a video controller. In one embodiment, adapters 27, 26, and 32 may be connected to one or more I/O busses that are connected to system bus 33 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 33 via user interface adapter 28 and display adapter 32. A keyboard 29, mouse 30, and speaker 31 all interconnected to bus 33 via user interface adapter 28, which may include, for example, a Super I/O chip integrating multiple device adapters into a single integrated circuit.

In exemplary embodiments, the processing system 300 includes a graphics processing unit 41. Graphics processing unit 41 is a specialized electronic circuit designed to manipulate and alter memory to accelerate the creation of images in a frame buffer intended for output to a display. In general, graphics processing unit 41 is very efficient at manipulating computer graphics and image processing and has a highly parallel structure that makes it more effective than general-purpose CPUs for algorithms where processing of large blocks of data is done in parallel.

Thus, as configured in FIG. 3, the system 300 includes processing capability in the form of processors 21, storage capability including system memory 34 and mass storage 24, input means such as keyboard 29 and mouse 30, and output capability including speaker 31 and display 35. In one embodiment, a portion of system memory 34 and mass storage 24 collectively store an operating system coordinate the functions of the various components shown in FIG. 3.

It is to be understood that the block diagram of FIG. 3 is not intended to indicate that the computer system 300 is to include all of the components shown in FIG. 3. Rather, the computer system 300 can include any appropriate fewer or additional components not illustrated in FIG. 3 (e.g., additional memory components, embedded controllers, modules, additional network interfaces, etc.). Further, the embodiments described herein with respect to computer system 300 may be implemented with any appropriate logic, wherein the logic, as referred to herein, can include any suitable hardware (e.g., a processor, an embedded controller, or an application specific integrated circuit, among others), software (e.g., an application, among others), firmware, or any suitable combination of hardware, software, and firmware, in various embodiments.

Turning now to an overview of technologies that are more specifically relevant to aspects of the invention, predicting patient outcomes can be a useful tool for physicians to make recommendations to a patient and educate the patient about how their everyday decisions can impact their long term health. Helping patients understand the impact that lifestyle changes can have in terms of long term outcomes with hard data about disease probabilities can be a powerful tool to assist with motivating change in the patient's behavior and habits. However, making said predictions can be difficult given the voluminous amount of unstructured medical data available.

Turning now to an overview of the aspects of the invention, one or more embodiments of the invention provide for a system for ingesting a large corpus of patient data and, for each patient, create temporal summary snapshots. For example, for a given patient history, summary snapshots can be created for different age groupings such as 25 years old, 30 years old, and a snapshot every 5 years thereafter can be taken. The snapshot can be summaries that include all manner of data such as, for example, current drugs taken, current diseases, length of time having the disease, lab results, lab results over time, genetic markets, and the like. These patient summary snapshots can be stored according to the time slice (e.g., age groupings) for each patient and cohorts can be created for each age group based on characteristics of the patient summary snapshots. For example, patients that are age 55 years old having a certain set of medical conditions, medications, behavior patterns, genetic markets, and the like can be grouped into a cohort.

When a healthcare professional wishes to determine or predict a current patient's future medical outcome, the current patient's medical history and current medical state can be ingested and summarized. Given a representation of this current patient, the system can find a similar patient cohort as a starting point for this current patient. Then, the patient cohort can be utilized to make predictions about the current patient's health outcomes at each time interval given the current patient's present health conditions. The predictions can be made initially given the patient makes no changes in their health behavior. These predictions can include, for example, current patient is 50% likely to be 20 lbs heavier in 5 years based on a comparison of the current patient's health condition and the stored patient cohort summaries described above. Another prediction could be new patient is 25% likely to develop fatty liver disease in 10 years based on the percentage of patients in the patient cohort that developed this disease in the time slice age grouping. In one or more embodiments of the invention, the system can also receive inputs for changing certain health behaviors to adjust the patient cohort at different time slice snapshots to predict how these changes to the health behaviors will impact their future healthcare outcomes. For example, if the current patient were to get a certain lab measurement down below a certain number, how would this change the patient cohort and thus the patient's predicted health.

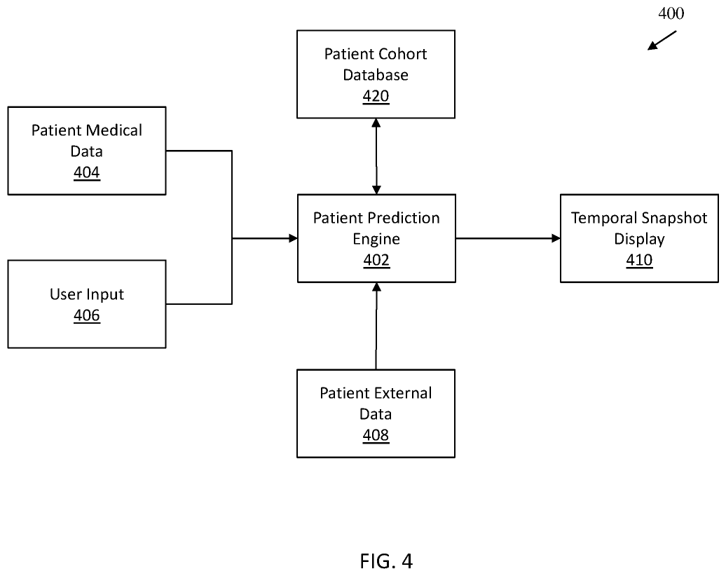

Turning now to a more detailed description of aspects of the present invention, FIG. 4 depicts block diagram of a system for dynamic patient outcomes predictions according to embodiments of the invention. In one or more embodiments of the invention, the system 400 includes a patient prediction engine 402 that is operable to create a patient cohort database 420 as briefly described above. The patient cohort database 420 includes snapshot summaries of patients taken from a large patient population at various age and population groups. For each patient from the large patient population data, snapshot summaries can be created for different age groups such, for example, every 2 years or every 5 years. These snapshot summaries created for each patient from the patient population data can be created, by the patient prediction engine 402, utilizing a variety of techniques including a fixed length vector patient summarization scheme such as, for example, a domain specific autoencoder. The domain specific autoencoder can be trained with relevant patient medical features (e.g., diseases, medications, etc.). The patient prediction engine 402 can implement an autoencoder by first taking in input data which can include, but is not limited to, pixel data, audio data, patient attributes, and the like. The input data is fed through an information bottleneck (i.e., a network layer that is smaller than the input layer). The learning objective for the network is simply to reproduce the original input. The bottleneck layer acts as a compressed encoding of the information that was fed into it (e.g., the fixed length vector of the input information). After being created, the snapshot summaries are stored in the patient cohort database 420. Cohort refers to groupings of similarly situated patients due to a variety of factors including, but not limited to, medical factors, behavioral factors, environmental factors, and/or genetic factors.

In one or more embodiments of the invention, the system 400 can be utilized to predict patient outcomes based on medical data received for a patient compared to the patient cohort data that includes snapshot summaries of a large population of patients. The patient prediction engine 402 can received patient medical data 404 for a given patient that is looking for predictive outcomes based on their current medical state. The patient medical data 404 is summarized using a summarization scheme and a patient cohort is identified for the patient. The patient cohort includes one or more other patients from the large patient population in the patient cohort database 420. The patient prediction engine 402 can then analyze cohort snapshot summaries of similarly situated patients in the cohort to generate predictive temporal snapshots for the patient based on the patient cohort. This can be displayed on a temporal snapshot display 410 for review by a medical professional and/or the patient. The predictive temporal snapshots include snapshot summaries at different advanced ages for the patient. For example, if a 25 year old patient is looking to determine their predictive temporal snapshot, the patient will submit his/her patient medical data 404 for analysis by the patient prediction engine 402. The patient prediction engine 402 determines a cohort for the patient (e.g., 25 year old patients with the same or similar medical conditions, patient characteristics, prescriptions, genetic data, patient behaviors, and the like). The cohort can be determined by comparing a summarization of the current patient to patient summarizations in the patient cohort database 420. This comparison can return this cohort using techniques and metrics such as, for example, cosign similarity. Cosine similarity can compare vectors in a geometric space. Other customer build classifiers can be utilized on top of patient embeddings like a K-nearest neighbor algorithm. The patient prediction engine 402 then pulls snapshot summaries for other patients in the same cohort to make predictions about the patient's future medical outcomes. The predictions can be risk levels for developing future diseases or risk levels for changes in patient's characteristics such as, for example, deteriorating mobility or increase in weight. These predictions are made at various time slices (age groups) in the patient cohort database 420. An example prediction would be a 50% chance of developing diabetes by age 50 years old based on the patient's current medical data and the cohort of patients at age 50 years old. This prediction would be based on the summaries of other patients in the patient cohort at each age group (i.e., time slice). In the above example, 50% of the other patients in the patient cohort developed diabetes by age 50 years old. Other metrics can be utilized for determining percentage likelihood including, but not limited to, weighted averages based on distance of vectors between patients in the vector space.

In one or more embodiments of the invention, the system 400 includes a user input 406 that can provide additional information and/or adjustments for the patient to the patient prediction engine 402. While above describes predictions for the patient based on their current state and current behavior/habits, the user input 406 allows for inputting adjustments to the user's medical data, behavior/habits, environmental data, and the like. For example, if the patient wishes to reduce the risk of a certain disease, the healthcare professional (or the patient) can make adjustments to a certain medical characteristic or behavior characteristic such as lowering the patient's average blood pressure, loosing a certain amount of weight, and/or changing jobs to reduce stress levels within a certain time period. These adjustments can change the patient cohort for the patient to show what changes in the patient's future healthcare outcomes are likely.

In one or more embodiments of the invention, the patient prediction engine 402 can also return a set of suggested changes for medical related characteristics or other changes to behavior or environmental changes. For example, if a patient wishes to change his/her cohort to be at less risk for a certain disease, the prediction engine 402 can identify cohorts that have less of a risk and determine medical characteristics and/or behavioral and environmental characteristics of other patients in this target cohort that can assist the current patient in changing cohorts so as to reduce their risk of a certain disease.

In one or more embodiments of the invention, the system 400 can obtain patient external data 408 from a variety of sources. The patient external data 408 can include data associated with a location of the patient's residence or where the patient works. This location data can be compared to data associated with environmental concerns such as environmental pollutants, smog, and the like which may make the patient more susceptible to certain diseases. This patient external data 408 can be utilized by the patient prediction engine 402 to further determine a proper patient cohort for the patient. In addition, the patient cohort database can also store external data about the other patients from the population data to further delineate cohorts based on, for example, proximity to environmental pollution and the like. The patient external data 408 can also include data associated with patient behaviors, habits, and environment such as work commute times each day, stress levels for the type of job the patient has, noise levels at the patient's residence, how much television the patient watches, activity levels of the patient, proximity to health food stores, and the like. This patient external data 408 can further be utilized for determining patient cohorts for predictive healthcare outcomes. Also, the suggested actions for the patient to change patient cohort to reduce certain healthcare risks can include changes to, for example, patient job, patient residence, patient commute times, and the like.

In one or more embodiments of the invention, the system 400 through the patient prediction engine 402 can generate interactions with external systems to assist a patient with changing their patient cohort. For example, if changing a cohort requires the patient change jobs or move to a residence with less noise level to reduce stress, the patient prediction engine 402 can post to a job posting website or search for a job posting website for the patient to find a less stressful job. Or the patient prediction engine 402 can search a real estate listing service or actively contact listings for purchasing or renting a residence in a quieter area or an area with less environmental pollution.

In one or more embodiments of the invention, the engine 402 can be implemented on the processing system 300 found in FIG. 3. Additionally, the cloud computing system 50 can be in wired or wireless electronic communication with one or all of the elements of the system 400. Cloud 50 can supplement, support or replace some or all of the functionality of the elements of the system 400. Additionally, some or all of the functionality of the elements of system 400 can be implemented as a node 10 (shown in FIGS. 1 and 2) of cloud 50. Cloud computing node 10 is only one example of a suitable cloud computing node and is not intended to suggest any limitation as to the scope of use or functionality of embodiments of the invention described herein.

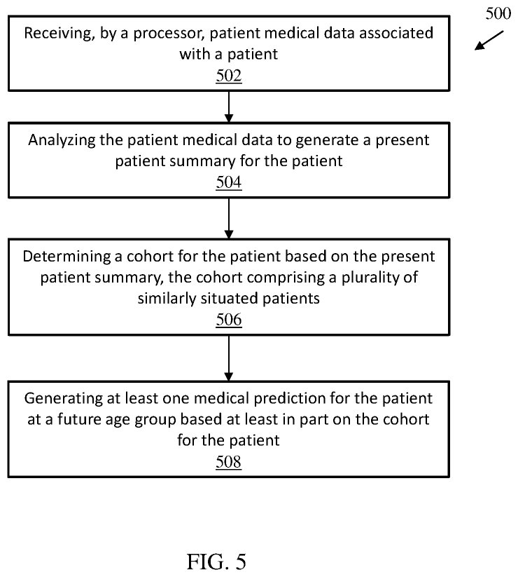

FIG. 5 depicts a flow diagram of a method for dynamic patient outcome predictions according to one or more embodiments of the invention. The method 500 includes receiving, by a processor, patient medical data associated with a patient, as shown at block 502. At block 504, the method 500 includes analyzing the patient medical data to generate a present patient summary for the patient. Also, the method 500, at block 506, includes determining a cohort for the patient based on the present patient summary, the cohort comprising a plurality of similarly situated patients. And at block 508, the method 500 includes generating at least one medical prediction for the patient at a future age group based at least in part on the cohort for the patient.

Additional processes may also be included. It should be understood that the processes depicted in FIG. 5 represent illustrations, and that other processes may be added or existing processes may be removed, modified, or rearranged without departing from the scope and spirit of the present disclosure.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
receiving, by a processor, patient medical data associated with a patient;
analyzing the patient medical data to generate a present patient summary for the patient;
determining a cohort for the patient based on the present patient summary, the cohort comprising a plurality of similarly situated patients; and
generating at least one medical prediction for the patient at a future age group based at least in part on the cohort for the patient.

2. The computer-implemented method of claim 1, further comprising:
determining a target cohort for the patient; and
analyzing a plurality of other patient summaries to determine one or more suggested medical changes for the patient to alter the cohort for the patient to the target cohort for the patient.

3. The computer-implemented method of claim 1, further comprising:
determining a target cohort for the patient; and
analyzing a plurality of other patient summaries to determine one or more suggested environmental changes for the patient to alter the cohort for the patient to the target cohort for the patient.

4. The computer-implemented method of claim 1, wherein determining the cohort for the patient based on the present patient summary comprises:
analyzing a plurality of other patient summaries taken at an age group of the patient; and
determining the cohort for the patient based on a similarity between the present patient summary and a set of similarly situated patient summaries taken at the age group of the patient.

5. The computer-implemented method of claim 4, wherein the similarity between the present patient summary and the set of similarly situated patient summaries comprises a cosign similarity metric.

6. The computer-implemented method of claim 4, wherein generating the at least one medical prediction for the patient at a future age group based at least in part on the cohort for the patient comprises:
analyzing a set of similarly situated patient summaries taken at the future age group; and
determining medical outcomes for the set of similarly situated patient summaries taken at the future age group to predict the at least one medical predictions for the patient at the future age group.

7. The computer-implemented method of claim 1, further comprising:
receiving a patient input comprising a modification of a behavior of the patient;
adjusting the patient cohort based on the modification of the behavior of the patient; and
generating a second medical prediction based on the adjustment to the patient cohort.

8. The computer-implemented method of claim 1, further comprising:
enacting an action based on the at least one medical prediction for the patient.

9. A system comprising:
a processor communicatively coupled to a memory, the processor configured to:
receive patient medical data associated with a patient;
analyze the patient medical data to generate a present patient summary for the patient;
determine a cohort for the patient based on the present patient summary, the cohort comprising a plurality of similarly situated patients; and
generate at least one medical prediction for the patient at a future age group based at least in part on the cohort for the patient.

10. The system of claim 9, wherein the processor is further configured to:
determine a target cohort for the patient; and
analyze a plurality of other patient summaries to determine one or more suggested medical changes for the patient to alter the cohort for the patient to the target cohort for the patient.

11. The system of claim 9, wherein the processor is further configured to:
determine a target cohort for the patient; and
analyze a plurality of other patient summaries to determine one or more suggested environmental changes for the patient to alter the cohort for the patient to the target cohort for the patient.

12. The system of claim 9, wherein determining the cohort for the patient based on the present patient summary comprises:
analyzing a plurality of other patient summaries taken at an age group of the patient; and
determining the cohort for the patient based on a similarity between the present patient summary and a set of similarly situated patient summaries taken at the age group of the patient.

13. The system of claim 12, wherein the similarity between the present patient summary and the set of similarly situated patient summaries comprises a cosign similarity metric.

14. The system of claim 12, wherein generating the at least one medical prediction for the patient at a future age group based at least in part on the cohort for the patient comprises:
analyzing a set of similarly situated patient summaries taken at the future age group; and
determining medical outcomes for the set of similarly situated patient summaries taken at the future age group to predict the at least one medical predictions for the patient at the future age group.

15. A computer program product for dynamic patient outcome predictions comprising a computer readable storage medium having program instructions embodied therewith, the program instructions executable by a processor to cause the processor to perform a method comprising:
receiving, by the processor, patient medical data associated with a patient;
analyzing the patient medical data to generate a present patient summary for the patient;
determining a cohort for the patient based on the present patient summary, the cohort comprising a plurality of similarly situated patients; and
generating at least one medical prediction for the patient at a future age group based at least in part on the cohort for the patient.

16. The computer program product of claim 15, further comprising:
determining a target cohort for the patient; and
analyzing a plurality of other patient summaries to determine one or more suggested medical changes for the patient to alter the cohort for the patient to the target cohort for the patient.

17. The computer program product of claim 15, further comprising:
determining a target cohort for the patient; and
analyzing a plurality of other patient summaries to determine one or more suggested environmental changes for the patient to alter the cohort for the patient to the target cohort for the patient.

18. The computer program product of claim 15, wherein determining the cohort for the patient based on the present patient summary comprises:
analyzing a plurality of other patient summaries taken at an age group of the patient; and
determining the cohort for the patient based on a similarity between the present patient summary and a set of similarly situated patient summaries taken at the age group of the patient.

19. The computer program product of claim 18, wherein the similarity between the present patient summary and the set of similarly situated patient summaries comprises a cosign similarity metric.

20. The computer program product of claim 18, wherein generating the at least one medical prediction for the patient at a future age group based at least in part on the cohort for the patient comprises:
analyzing a set of similarly situated patient summaries taken at the future age group; and
determining medical outcomes for the set of similarly situated patient summaries taken at the future age group to predict the at least one medical predictions for the patient at the future age group.
